(19) United States
(12) Patent Application Publication
(10) Pub. No.: US 2025/0244335 A1
(43) Pub. Date: Jul. 31, 2025

(54) METHOD FOR HOST CELL PROTEIN IDENTIFICATION IN BIOTHERAPEUTIC SAMPLES

(71) Applicant: Regeneron Pharmaceuticals, Inc., Tarrytown, NY (US)

(72) Inventors: Shihan Huo, Tarrytown, NY (US); Song Nie, Armonk, NY (US); Shunhai Wang, Scarsdale, NY (US); Ning Li, New Canaan, CT (US)

(21) Appl. No.: 19/036,499

(22) Filed: Jan. 24, 2025

Related U.S. Application Data
(60) Provisional application No. 63/625,562, filed on Jan. 26, 2024.

(51) Int. Cl. G01N 33/68

(52) U.S. Cl. CPC G01N 33/6848; G01N 2333/015

(57) ABSTRACT

The present invention provides methods for identifying, quantifying, and/or characterizing at least one host cell protein (HCP) impurity in a sample containing AAV vectors. The HCP impurities can be identified through digesting the sample generating peptides which can be subsequently purified through the use of paramagnetic beads. The purified peptides can then be subjected to liquid chromatography-mass spectrometry (LC-MS) utilizing wide window acquisition to identify, quantify and/or characterize the at least one HCP impurity. In addition, the sample can be purified using filter-aided sample preparation to purify the digested proteins prior to LC-MS.

### CROSS REFERENCE TO RELATED APPLICATIONS

This application claims the benefit of priority from U.S. Provisional Application No. 63/625,562, filed on Jan. 26, 2024, which is herein incorporated by reference in its entirety.

### FIELD OF THE DISCLOSURE

The present disclosure generally relates to methods of identifying, quantifying, and/or characterizing at least one host cell protein (HCP) impurity in a sample containing a biotherapeutic.

### BACKGROUND

Gene therapy biopharmaceuticals mediate therapeutic effects by transcription and/or translation of transferred genetic material, such as integrating genetic material into the host genome, and are used to treat, prevent or cure a disease. Currently, gene therapy is one of the most investigated therapeutic modalities in preclinical and clinical settings. However, gene therapy experienced a major setback in the late 1990's and early 2000's which raised concerns about the safety of gene therapy and highlighted the critical need for safer gene delivery vectors. A better understanding of gene delivery vectors and advancing the manufacture of safe and effective vectors is helpful to mitigate safety risks.

Gene delivery vectors are important to ensure efficient gene delivery to the target tissue and cells. The ideal gene delivery system should have high gene transfer efficiency, low toxicity to the cell, and single cell specificity to the intended target. Based on gene delivery vector types, vectors can be divided into non-viral vectors and viral vectors. Due to the high gene transfer efficiency of viral vectors, they have been widely used in clinical trials.

Adeno-associated virus (AAV) is the most widely used viral vector for in vivo gene therapy applications. AAVs have low immunogenicity and can enable long-term, stable gene expression. The use of AAVs for gene therapy has created the need for analytical methods to monitor and characterize these products. Process-related and product-related impurities should be monitored to ensure product quality and process consistency.

Biopharmaceutical products must meet very high standards of purity. Thus, it is important to monitor any impurities in such biopharmaceutical products at different stages of drug development, production, storage and handling. Residual impurities should be at an acceptable low level prior to conducting clinical studies. Residual impurities are also a concern for biopharmaceutical products intended for end-users. For example, host cell proteins (HCPs) can be present in biopharmaceuticals which are developed using cell-based systems. The presence of HCPs in drug products should be monitored and can be unacceptable above a certain threshold, depending on the product and the particular HCP. Sometimes, even trace amounts of HCPs can cause an immunogenic response in an end-user.

It will be appreciated that a need exists for methods to identify, characterize and quantitate (quantify) HCPs to monitor and control the residual HCPs in a drug substance, biopharmaceutical product, or other product to mitigate safety risks.

### SUMMARY

This disclosure provides methods of identifying, quantifying and/or characterizing HCP impurities in a sample. In some embodiments, the sample contains a biotherapeutic, such as a viral particle or vector (e.g., AAV vector), antibody (e.g., a monoclonal antibody or “mAb”), enzyme, cytokine, or growth factor, along with an HCP in low abundance relative to the biotherapeutic.

In one aspect, the present disclosure is directed to a method of identifying, quantifying and/or characterizing at least one HCP impurity in a sample containing a biotherapeutic (e.g., AAV vector), wherein the method includes the following steps: (a) treating such a sample to enzymatic digestion to produce a peptide digest; (b) subjecting the peptide digest to paramagnetic beads to form a purified peptide digest; and (c) subjecting the purified peptide digest to liquid chromatography-mass spectrometry (LC-MS) analysis utilizing wide window acquisition (WWA) to identify, quantify, and/or characterize the at least one HCP impurity.

In some embodiments, the mass spectrometry is tandem mass spectrometry. In some embodiments, the resolution of the tandem mass spectrometry is about 15K to about 120K. In some embodiments, the resolution of the tandem mass spectrometry is about 60K. In some embodiments, the isolation window of the tandem mass spectrometry is between about one mass-to-charge ratio and about 18 mass-to-charge ratio. In some embodiments, the isolation window of the tandem mass spectrometry is about 4 mass-to-charge ratio.

In some embodiments, the sample amount is from about 1 μg to about 10 μg. In some embodiments, the sample amount is about 2.5 μg.

In some embodiments, the AAV vector comprises a serotype selected from a group comprising of AAV1, AAV2, AAV3, AAV4, AAV5, AAV6, AAV7, AAV8, AAVrh8, AAV2/8, AAV9, AAV10, AAV11, AAV12, variations thereof and combinations thereof.

In some embodiments, the HCP impurity is a peptide impurity. In some embodiments, peptide impurity identification is enhanced by a search algorithm. In some embodiments, the search algorithm is CHIMERYS™.

In some embodiments, the paramagnetic beads are used in a single-pot, solid-phase enhanced sample-preparation (SP3) process.

In another aspect, the present disclosure is directed to a method of identifying, quantifying and/or characterizing at least one HCP impurity in a sample containing a biotherapeutic (e.g., AAV vector or mAb), wherein the method includes the following steps: (a) treating such a sample to enzymatic digestion to produce a peptide digest; (b) subjecting said peptide digest to filter-aided sample preparation (FASP) to form a purified peptide digest; and (c) subjecting the purified peptide digest to liquid chromatography-mass spectrometry (LC-MS) analysis utilizing wide window acquisition (WWA) to identify, quantify, and/or characterize said at least one HCP impurity.

In some embodiments, the mass spectrometry is tandem mass spectrometry. In some embodiments, the resolution of the tandem mass spectrometry is about 15K to about 120K. In some embodiments, the resolution of the tandem mass spectrometry is about 60K. In some embodiments, the isolation window of the tandem mass spectrometry is between about one mass-to-charge ratio and about 18 mass-to-charge ratio. In some embodiments, the isolation window of the tandem mass spectrometry is about 4 mass-to-charge ratio.

In some embodiments, the sample amount is from about 1 μg to about 10 μg. In some embodiments, the sample amount is about 2.5 μg.

The biotherapeutic may be, for example, a viral particle or vector (e.g., AAV vector), antibody, enzyme, cytokine, or growth factor. In some embodiments, the AAV vector comprises a serotype selected from a group comprising of AAV1, AAV2, AAV3, AAV4, AAV5, AAV6, AAV7, AAV8, AAVrh8, AAV2/8, AAV9, AAV10, AAV11, AAV12, variations thereof and combinations thereof.

In some embodiments, the HCP impurity is a peptide impurity. In some embodiments, peptide impurity identification is enhanced by a search algorithm. In some embodiments, the search algorithm is CHIMERYS™.

In another aspect, the present disclosure is directed to a method of identifying, quantifying and/or characterizing HCP impurities in a sample containing a monoclonal antibody (mAb) as the biotherapeutic, wherein the method includes the following steps: (a) treating such a sample to enzymatic digestion to produce a peptide digest; and (b) subjecting said purified peptide digest to liquid chromatography-mass spectrometry (LC-MS) analysis utilizing wide window acquisition (WWA) to identify, quantify, and/or characterize the at least one HCP impurity.

In some embodiments, the mass spectrometry is tandem mass spectrometry. In some embodiments, the resolution of the tandem mass spectrometry is about 15K to about 120K. In some embodiments, the resolution of the tandem mass spectrometry is about 60K. In some embodiments, the isolation window of the tandem mass spectrometry is between about one mass-to-charge ratio and about 18 mass-to-charge ratio. In some embodiments, the isolation window of the tandem mass spectrometry is about 4 mass-to-charge ratio.

In some embodiments, the sample amount is from about 500 μg to about 3 mg. In some embodiments, the sample concentration is about 1 mg.

In some embodiments, the HCP impurity is a peptide impurity. In some embodiments, peptide impurity identification is enhanced by a search algorithm. In some embodiments, the search algorithm is CHIMERYS™.

In another aspect, the present disclosure is directed to a method of identifying, quantifying and/or characterizing at least one HCP impurity in a sample, wherein the method includes the following steps: (a) treating such a sample to enzymatic digestion to produce a peptide digest; (b) subjecting said peptide digest to a single-pot, solid-phase enhanced sample-preparation paramagnetic beads to form a purified peptide digest; and (c) subjecting said purified peptide digest to liquid chromatography-mass spectrometry (LC-MS) analysis utilizing wide window acquisition (WWA) to identify, quantify, and/or characterize the at least one HCP impurity.

In some embodiments, the mass spectrometry is tandem mass spectrometry. In some embodiments, the resolution of the tandem mass spectrometry is about 15K to about 120K. In some embodiments, the resolution of the tandem mass spectrometry is about 60K. In some embodiments, the isolation window of the tandem mass spectrometry is between about one mass-to-charge ratio and about 18 mass-to-charge ratio. In some embodiments, the isolation window of the tandem mass spectrometry is about 4 mass-to-charge ratio.

In some embodiments, the sample amount is from about 1 μg to about 10 μg. In some embodiments, the sample amount is about 2.5 μg.

In some embodiments, the AAV vector comprises a serotype selected from a group comprising of AAV1, AAV2, AAV3, AAV4, AAV5, AAV6, AAV7, AAV8, AAVrh8, AAV2/8, AAV9, AAV10, AAV11, AAV12, variations thereof and combinations thereof.

In some embodiments, the HCP impurity is a peptide impurity. In some embodiments, peptide impurity identification is enhanced by a search algorithm. In some embodiments, the search algorithm is CHIMERYS™.

These, and other, aspects of the present invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the present invention.

### BRIEF DESCRIPTION OF THE DRAWINGS

FIG. 1 illustrates three different sample purification methods used during sample preparation and the comparison of the number of proteins identified, according to an exemplary embodiment.

FIG. 2 shows a comparison of two sample purification methods, SP3 and FASP, with varying amounts of protein input, according to an exemplary embodiment.

FIG. 3 shows the comparison of the precision of two sample purification methods, SP3 and FASP, with varying amounts of protein input, according to an exemplary embodiment.

FIG. 4 shows the comparison of a mass spectrum generated from a traditional search algorithm with only one peptide-to-spectrum matching event to the mass spectrum generated from a search algorithm using wide window acquisition with a three peptide-to-spectrum matching events, according to an exemplary embodiment.

FIG. 5A shows the comparison of MS2 resolution and the number of identified proteins using NIST mAb, according to an exemplary embodiment.

FIG. 5B shows the comparison of the MS2 isolation window and the number of identified proteins using NIST mAb, according to an exemplary embodiment.

FIG. 5C shows the overall increase in the number of proteins identified comparing traditional data dependent acquisition and wide window acquisition, according to an exemplary embodiment.

FIG. 5D illustrates the comparison of proteins identified by traditional data dependent acquisition and wide window acquisition, according to an exemplary embodiment.

FIGS. 6A and 6B show the comparison of MS2 resolution (FIG. 6A) or MS2 isolation window (FIG. 6B) and the number of identified proteins using AAV1, according to an exemplary embodiment.

FIG. 7 shows the chimeric mass spectrum generated using the optimized wide window acquisition method of the present invention, according to an exemplary embodiment.

FIG. 8 shows the comparison of the number of peptide-to-spectrum matches for traditional data dependent acquisition and wide window acquisition, according to an exemplary embodiment.

FIG. 9 shows the overall increase in the percentage of identified proteins comparing traditional data dependent acquisition and the optimized wide window acquisition of the present invention, according to an exemplary embodiment.

FIG. 10 shows the comparison of conditions tested using the method of the present invention, according to an exemplary embodiment.

FIG. 11 shows the number of peptides identified using three different AAV serotypes comparing direct digestion, traditional data dependent acquisition and SEQUEST search engine (control) to the method of the present invention using SP3 digestion, wide window acquisition and CHIMERYS™ search algorithm (optimized), according to an exemplary embodiment.

FIG. 12 shows the comparison of the number of identified proteins changing one variable (sample preparation method, acquisition method, and search engine/algorithm) at a time, according to an exemplary embodiment.

FIG. 13 shows the sequence coverage of three different AAVs comparing the direct digestion, traditional data dependent acquisition (control) to the method of the present invention using SP3 digestion and wide window acquisition, both were processed using Byonic software, according to an exemplary embodiment.

FIG. 14 shows high-risk HCPs identified in commercial AAV1 using the method of the present invention, according to an exemplary embodiment.

FIG. 15 shows viral protein contaminants identified in AAVs, according to an exemplary embodiment.

### DETAILED DESCRIPTION

AAVs have been widely used as gene delivery vectors to deliver genetic material, such as delivering nucleic acids for gene therapy. AAVs provide the advantages of non-pathogenicity and low immunogenicity. AAVs are nonpathogenic members of the Parvoviridae family under Dependovirus genus and require helpers, such as Adenovirus or Herpesvirus, for infection (Venkatakrishnan et al., Structure and Dynamics of Adeno-Associated Virus Serotype 1 VP1-Unique N-Terminal Domain and Its Role in Capsid Trafficking, Journal of Virology, May 2013, vol. 87, no. 9, pages 4974-4984). AAV encapsulates a single-stranded DNA genome of about 4.8 kilobases (kb) in an icosahedral capsid which is made of a shell of capsid viral proteins. Recombinant AAV genomes are nonpathogenic and do not integrate into a host's genome, but exist as stable episomes that provide long-term expression. AAV serotypes make a very useful system for preferentially transducing specific cell types.

Overall, AAV-based therapy has the advantages of being non-pathogenic and non-toxic, having cell type-specific infection, and offering different serotypes with varying cell transduction efficiencies. A disadvantage is that AAV production, purification, and characterization are more complex compared to, for example, antibody therapies. Fully packaged AAVs consist of an icosahedral capsid containing an about 4.8 kb single-stranded genome. An empty capsid has a molecular weight of about 3750 kDa, while a full capsid with an about 4.7 kb single-stranded genome has a molecular weight of about 5100 kDa. The purity of AAVs is defined by several product-related impurities, including empty capsids, capsids containing partial or incorrect genomes, and aggregated or degraded capsid, as well as residual HCPs.

HCPs are protein impurities that originate from the host cells utilized during the upstream biotherapeutic production. Despite necessary and extensive downstream purification, trace levels of residual HCPs remain in the final drug product. Some of these HCPs can potentially cause drug aggregation, fragmentation, modification, or even trigger immunogenic responses in patients. Therefore, it is crucial to have highly sensitive and reproducible methods for constant monitoring and evaluation of residual HCPs for drug quality control. For monoclonal antibody (mAb) drugs, analyzing HCPs using the LC-MS method often presents a challenge due to the high dynamic range (often>5-6 orders of magnitude) between low-level HCPs and high-level mAb. Numerous methods have been established for HCP analysis in mAb drug products, including native digestion, low enzyme differentiation digestion, affinity enrichment of HCPs, or mAb depletion. These methods have achieved tremendous success and allow the detection of many low abundance HCPs with sub-ppm sensitivity.

However, for newer modality biotherapeutics, such as AAVs, these methods cannot be easily adopted. This is primarily due to the limited availability of AAV samples, in addition to the presence of surfactants in the final AAV product, which necessitates a different approach for sample preparation. The smaller starting material used for AAV products, at the microgram level, also prohibits the use of typical enrichment or depletion methods. The similar high dynamic range issue in AAV samples requires efforts to develop and evaluate newer LC-MS methods, which are important but often overlooked. Finally, the different production systems of AAVs are complex and the involvement of other proteins, such as viral proteins, have not been comprehensively evaluated. Therefore, the need to assess viral proteins is important.

Recently, Smith et al. reported the use of single-pot, solid-phase-enhanced sample-preparation (SP3) for AAV sample preparation for HCP analysis, resulting in the detection of many HCPs with a 10 μg protein input. However, a lower protein input was not evaluated. Additionally, advancements in mass spectrometry technology, such as the rapid progress of single-cell analysis, have led to the development of new LC-MS approaches to increase sensitivity.

One such method is the use of a wider precursor isolation window during data dependent acquisition (DDA) followed by the use of a search algorithm, such as the CHIMERYS™ search algorithm, for additional peptide identification from many existing chimeric spectra. This method of the present invention has proven effective in single-cell analysis due to the very limited sample input with low ion flux. Therefore, wide window acquisition and utilizing a CHIMERYS™ search algorithm to deconvolute the spectra was used to mitigate the high dynamic range issue in HCP analysis.

In order to identify HCPs, several approaches have been employed, such as gel electrophoresis and/or digestion coupled with liquid chromatography-mass spectrometry (LC-MS), or digestion followed by enrichment with liquid chromatography (LC) followed by LC-MS. The use of LC-MS for HCP profiling offers an alternative analytical approach to ELISA, thus mitigating the risk associated with antibody coverage. LC-MS allows for the identification of individual HCPs and facilitates monitoring of low abundance HCPs. A major challenge of HCP profiling by mass spectrometry is that the dynamic range between low abundance HCP and the drug substance is beyond the range of most current mass spectrometry.

For therapeutic protein HCP analysis, multiple methods have been developed to overcome the dynamic range issues. The residual HCPs in therapeutic proteins are enriched through molecular weight cutoff filtration, polyclonal antibodies capture, or removal of the therapeutic protein with affinity purification using Protein A or Protein G. Recently, the native digestion method has gained popularity in monoclonal antibody (mAb) HCP analysis, because of its high sensitivity and simple workflow. However, these profiling methods for therapeutic protein HCPs are not readily applicable to the monitoring of HCPs in AAV. Most highly sensitive methods require several or even hundreds of milligrams of therapeutic protein for sample preparation. Such an amount is not feasible for AAV, because of the limited quantities of available samples. Moreover, AAVs exhibit distinct characteristics compared to therapeutic proteins, thereby posing a hurdle to the direct application of mAb analysis methods to AAV. To date, AAV drug substance HCP analysis by LC-MS has been accomplished through direct digestion, gel-electrophoresis fractionation and SP3 methods. These methods either require extensive HCP fractionation steps or lack the sensitivity to detect low abundance HCPs. However, the dynamic concentration of HCPs in the AAVs, the limitations of sample size and AAV concentration are major challenges to monitor and remove HCP impurities. Therefore, the need for a simple and sensitive AAV HCP LC-MS profiling method persists.

The present disclosure provides methods to identify, characterize and/or quantitate HCPs using an enhanced sample preparation method and an optimized wide window acquisition method. In some exemplary embodiments, the enhanced sample preparation method and the wide window acquisition method permits the detection of HCPs when lower protein or AAV concentrations are used. The methods disclosed herein overcome longstanding problems in identifying HCPs in biopharmaceutical products.

In the method, the sample is subjected to peptide enzymatic digestion to produce a peptide digest. Methods for peptide enzymatic digestion are well known in the art. The digestion is typically effected by a protease, such as trypsin. The digestion is preferably a direct digestion process.

In a next step of the method, the peptide digest is subjected to a purification process to form a purified peptide digest. Such methods are well known in the art. For example, in some embodiments, the peptide digest is contacted with specially functionalized paramagnetic beads that have a high affinity for peptide products, as well known in the art. In some embodiments, the paramagnetic beads may be used in a single-pot, solid phase-enhanced sample-preparation (SP3) process, as well known in the art. The SP3 process is described in detail in C. S. Hughes et al., Nature Protocols, 2019, 14 (1), pp. 68-85, the contents of which are herein incorporated by reference. To induce protein binding to beads, an equal volume of ethanol may be added to the sample to achieve a final concentration of ethanol of 50%. The mixture may then be incubated at ambient temperature (e.g., 20-25° C.) for 10-15 minutes at a suitable speed (e.g., 500-2000 rpm). The beads may then be held by an external magnet will the supernatant is discarded, followed by washing of the beads (e.g., two or three times with 50-80% ethanol) and elution to obtain the peptide digestion products separated from the beads. Notably, the SP3 technique is also highly effective in removing surfactants through facile washing steps with a high organic solvent, followed by on-bead protein digestion. Thus, in some embodiments, the beads may be first contacted with the sample to selectively bind protein/peptide species to the beads, followed by on-bead protein/peptide digestion. Post-digestion, the sample may be centrifuged at about 20,000 g for about 1 min. The supernatant may then be collected on a magnetic rack and transferred to a new tube for reaction termination and subsequent LC-MS analysis. By another exemplary method, the peptide digest is treated by Filter-Aided Sample Preparation (FASP) to form a purified peptide digest. As well known in the art, the FASP method utilizes a filtration step for removal of salts and surfactant (aided by urea), followed by on-filter digestion. The FASP method is described in further detail in, for example, Wiśniewski, J. R., et al., Nat. Methods, 2009, 6 (5), pp. 359-362, the contents of which are herein incorporated by reference. A significant increase in the percentage of identified proteins was herein observed by use of the FASP and SP3 sample preparation methods.

In a next step of the method, the purified peptide digest is subjected to liquid chromatography-mass spectrometry (LC-MS) analysis utilizing wide window acquisition to identify, quantify, and/or characterize said at least one HCP impurity. The MS is preferably tandem MS, as further described below. As further discussed below, the resolution and isolation window of the tandem MS method (among other parameters) may be particularly selected, such as disclosed anywhere in this disclosure, to permit the identification, quantification, and/or characterization of at least HCP impurity that is present in low abundance relative to the biotherapeutic.

A significant advantage of the presently described method is that it permits the mass of the sample (i.e., “sample amount”) to be substantially lower than sample amounts conventionally used in the art. While the conventional art may require sample amounts in the tens or hundreds of milligrams, the sample amount that can be used in the presently described method may be substantially less than 1 mg or even 0.001 mg. By virtue of the present method, the sample amount may be, for example, in a range of about 1 μg to about 20 μg. In different embodiments, the sample amount is precisely or about 1 μg, 2.5 μg, 5 μg, 7.5 μg, 10 μg, 12 μg, 15 μg, or 20 μg, or a mass within a range between any of these values, e.g., 1-20 μg, 2.5-20 μg, 5-20 μg, 7.5-20 μg, 10-20 μg, 1-12 μg, 2.5-12 μg, 5-12 μg, 7.5-12 μg, 1-10 μg, 2.5-10 μg, 5-10 μg, 7.5-10 μg, 1-7.5 μg, 2.5-7.5 μg, or 1-5 μg. In some embodiments, a low AAV input of 2.5 μg may be utilized for the HCP analysis. In some embodiments, a low AAV input is equivalent to a viral copy number of about 1.0×10¹¹ to 10×10¹¹ per sample, e.g., 4.0×10¹¹ per sample. In some embodiments, the specific selection of the resolution and isolation window of the tandem MS method, such as disclosed anywhere in this disclosure, permits the use of such low sample amounts.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins may include one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may contain one or multiple polypeptides to form a single functioning biomolecule.

As used herein, the term “therapeutic protein” includes proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies.

In some embodiments, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., 28 BIOTECHNOLOGY AND GENETIC ENGINEERING REVIEWS 147-176 (2012), the entirety of which is herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments, the antibody molecule is a full-length antibody (e.g., an IgG1) or alternatively the antibody can be a fragment (e.g., an Fc fragment or a Fab fragment).

The term “antibody,” as used herein, includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” (bsAbs) includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or kx-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific are also contemplated.

The term “monoclonal antibody” (i.e., “mAb”), as used herein, is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

As used herein, a “protein pharmaceutical product” or “biopharmaceutical product” includes an active ingredient which can be fully or partially biological in nature. In some embodiments, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In some embodiments, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

As used herein, a “sample” refers contains at least a biotherapeutic substance and at least one host cell protein (HCP) impurity, wherein the HCP is present in low abundance relative to the biotherapeutic. The term “biotherapeutic” refers to any substance that has a therapeutic effect on a living organism, wherein the biotherapeutic may be natural or artificial. The biotherapeutic is typically nucleotide- or peptide-based. Some examples of biotherapeutics include a viral vector (e.g., AAV vector), monoclonal antibody (mAb), enzyme, cytokine, or growth factor. In some embodiments, the sample includes a mixture of molecules that includes at least a viral particle, such as an AAV particle, or an empty viral capsid. The sample is subjected to manipulation in accordance with the methods of the invention, including, for example, separating, analyzing, extracting, concentrating, profiling and the like.

As indicated above, the biotherapeutic substance is present in the sample in high abundance while the HCP is present in the sample in a low abundance. In some exemplary embodiments, a concentration of the at least one biotherapeutic can be at least about 1000 times, about 10,000 times, about 100,000 times or about 1,000,000 times higher than a concentration of the at least one HCP. Conversely, a concentration of the low-abundance HCP can be at or below 10-3, 104, 105, or 106 of the concentration of the biotherapeutic. Another way of expressing the relative concentrations is, for example, in parts per million (ppm). It should be understood that when using ppm to describe the concentration of a low-abundance protein or peptide, such as an HCP, in a sample that includes a high-abundance protein or peptide, such as a therapeutic nucleotide or protein, ppm is measured relative to the concentration of the high-abundance protein or peptide. The low-abundant amount of HCP may alternatively be provided in non-relative (i.e., absolute) terms, such as a concentration of no more than or less than 1000 ppm, 100 ppm, 10 ppm, or 1 ppm.

As used herein, the term “impurity” can include any undesirable protein present in a protein sample or protein biopharmaceutical product. Impurities can include process and product-related impurities. The impurity can further be of known structure, partially characterized, or unidentified. Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

As used herein, the term “host-cell protein” (HCP) includes protein derived from a host cell. Host-cell proteins can be a process-related impurity which can be derived from the manufacturing process. In some exemplary embodiments, the types of HCP process-related impurities in the composition can be at least two.

The presence of a host cell protein in a biotherapeutic product may be considered to be a higher or lower risk based on a number of measurable factors. One such factor is the concentration or abundance (quantity) of an HCP impurity in a biotherapeutic product. An HCP may have no discernible impact at a low enough abundance, as measured by, for example, ELISA or mass spectrometry. The level at which an HCP may present a considerable risk, which may be considered an unacceptable level in a product and may be monitored as a critical quality attribute (CQA), may depend on the specific identity of the HCP. Particular HCPs may be known to present a risk at a particular level, for example depending on the level of enzymatic activity of an HCP that is an enzyme.

Relatedly, the criticality of the presence of an HCP may depend on the function of that HCP, in particular relation to the components of the biotherapeutic product. For example, an HCP lipase that may or is known to degrade polysorbate that is present in the biotherapeutic product of interest may be closely monitored and may have a low threshold for how much of the HCP impurity can be allowed in the biotherapeutic product. Other HCPs of particular concern may be, for example, proteases that may or are known to degrade a protein of interest in the biotherapeutic product, or immunogenic HCPs that may or are known to cause an immune reaction when administered to a subject. Using the method of the present invention, a person skilled in the art may evaluate the abundance, distribution, and/or identity of an HCP impurity in the context of the biotherapeutic product of interest to determine if the HCP impurity is an HCP impurity of concern, and based on that determination may use chromatographic or other separation methods to remove the impurity when producing the biotherapeutic product.

The terms “peptide,” “protein” and “polypeptide” refer, interchangeably, to a polymer of amino acids and/or amino acid analogs that are joined by peptide bonds or peptide bond mimetics. The twenty naturally-occurring amino acids and their single-letter and three-letter designations are as follows: Alanine A Ala; Cysteine C Cys; Aspartic Acid D Asp; Glutamic acid E Glu; Phenylalanine F Phe; Glycine G Gly; Histidine H His; Isoleucine I He; Lysine K Lys; Leucine L Leu; Methionine M Met; Asparagine N Asn; Proline P Pro; Glutamine Q Gln; Arginine R Arg; Serine S Ser; Threonine T Thr; Valine V Val; Tryptophan w Trp; and Tyrosine Y Tyr.

As used herein, the term “vector” refers to a recombinant plasmid or virus (“viral vector”) that comprises a nucleic acid to be delivered into a host cell, either in vitro or in vivo. For example, in the context of AAV vectors, the term “vector” can be understood as referring to a vehicle or carrier used to deliver genetic material into cells. AAV vectors are commonly used in gene therapy to introduce therapeutic genes into a patient's cells to treat genetic disorders. AAV (i.e., viral particle) is assembled by 60 VP (viral protein) subunits forming a capsid that encapsulates a single-stranded DNA (ssDNA) genome, which contains AAV nucleic acids and target gene. Thus, a “viral vector” such as “an AAV vector”, as referred to herein, includes target nucleic acids, as well as viral particles. Vectors derived from AAV are particularly attractive for delivering genetic material because (i) they are able to infect (transduce) a wide variety of non-dividing and dividing cell types including muscle fibers and neurons; (ii) they may be made devoid of the virus structural genes, thereby eliminating the natural host cell responses to virus infection, for example, interferon-mediated responses; (iii) wild type AAVs have not been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected).

A “recombinant viral vector” refers to a recombinant polynucleotide vector including one or more heterologous sequences (e.g., nucleic acid sequence not of viral origin), and to a recombinant viral particle.

A “recombinant AAV vector (rAAV vector)” refers to a polynucleotide vector including one or more heterologous sequences (e.g., nucleic acid sequence not of AAV origin) that may be flanked by at least one, or for example, two, AAV inverted terminal repeat sequences (ITRs), as well as to a recombinant AAV viral particle. rAAV polynucleotide vectors can be replicated and packaged into infectious viral particles when present in a host cell that has been infected with a suitable helper virus (or that is expressing suitable helper functions) and that is expressing AAV rep and cap gene products (e.g., AAV Rep and Cap proteins).

A “capsid” is the protein shell of a virus, which encloses the genetic material. Three viral capsid proteins, VP1, VP1 and VP3, form the viral icosahedral capsid of 60 subunits in a ratio of 1:1:10. A full capsid contains genetic material and is required to provide therapeutic benefit. An empty capsid lacks the genome and therefore lacks the ability to provide therapeutic benefit to the patient.

A “viral particle” refers to a particle composed of at least one viral capsid protein and an encapsulated viral genome. While AAV is described in this disclosure as a model virus or viral particle, it is contemplated that the disclosed methods can be applied to profile a variety of viruses, e.g., the viral families, subfamilies, and genera. In some embodiments, the viral capsid, virus, or viral particle belongs to a viral family selected from the group consisting of Adenoviridae, Parvoviridae, Retroviridae, Baculoviridae, and Herpesviridae. In some embodiments, the viral capsid, virus, or viral particle belongs to a viral genus selected from the group consisting of Atadenovirus, Aviadenovirus, Ichtadenovirus, Mastadenovirus, Siadenovirus, Ambidensovirus, Brevidensovirus, Hepandensovirus, Iteradensovirus, Penstyldensovirus, Amdoparvovirus, Aveparvovirus, Bocaparvovirus, Copiparvovirus, Dependoparvovirus, Erythroparvovirus, Protoparvovirus, Tetraparvovirus, Alpharetrovirus, Betaretrovirus, Deltaretrovirus, Epsilonretrovirus, Gammaretrovirus, Lentivirus, Spumavirus, Alphabaculovirus, Betabaculovirus, Deltabaculovirus, Gammabaculovirus, Iltovirus, Mardivirus, Simplexvirus, Varicellovirus, Cytomegalovirus, Muromegalovirus, Proboscivirus, Roscolovirus, Lymphocryptovirus, Macavirus, Percavirus, and Rhadinovirus.

As used herein, the term “gene therapy” is a method of treatment of a genetic disease by modifying or manipulating a gene of interest. The key step in gene therapy is efficient delivery of a vector to the appropriate tissue or cells. Non-limiting examples of gene therapy products include plasmid DNA, viral vectors, non-viral vectors, bacterial vectors, human gene editing technology, and patient-derived cellular gene therapy products.

In some exemplary embodiments, the sample can be prepared prior to LC-MS analysis. Preparation steps can include reduction, denaturation, alkylation, dilution, digestion, and separation (for example, centrifugation).

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

Proteins may have distinctive susceptibility to denaturation. For example, viral proteins assembled into a viral capsid may have reduced susceptibility to denaturation compared to a monomeric or smaller multimeric protein, for example a host cell protein. A difference in susceptibility to denaturation may be taken advantage of in order to preferentially denature a particular protein or class of proteins while leaving another protein or class of proteins in a substantially natively folded state. In some embodiments, denaturation may be performed at a temperature selected to substantially denature a protein or class of proteins, such as, for example, non-viral proteins, such as host cell proteins, while leaving the proteins of a viral capsid substantially in a folded state. This may be referred to as, for example, mild denaturation, limited denaturation, partial denaturation, or differential denaturation. A partially denatured sample may then be subjected to a digestion step to produce a peptide digest. Because denatured proteins are more susceptible to digestion by digestive enzymes, the peptide digest will preferentially include peptides from the more denatured proteins, such as, for example, non-viral proteins, such as HCPs, compared to peptides from the more natively folded proteins, for example viral capsid proteins. This may be referred to as, for example, partial digestion, differential digestion, or limited digestion. The peptide digest will be enriched for peptides of the non-viral protein, such as HCPs, relative to the original sample. This enrichment may be useful for subsequent analysis, for example liquid chromatography-mass spectrometry analysis, in order to sensitively and accurately identify, characterize, and quantify the non-viral proteins.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” (i.e., “peptide digestion products”) that can be further analyzed using peptide mapping analysis.

As used herein, the term “digestive enzyme” refers to any of a large number of enzymes that can perform digestion of a protein. Non-limiting examples of hydrolyzing enzyme that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinas Arg-C(Arg-C), endoproteinase Glu-C(Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, 12 JOURNAL OF PROTEOME RESEARCH 1067-1077 (2013)).

Conventional methods use a digestive enzyme under conditions and concentrations sufficient to completely digest all protein in a sample prior to LC-MS analysis. However, the present disclosure may identify and quantify low-abundance proteins, such as HCPs, by a limited digestion, which means that denaturation and digestion conditions are selected such that proteins in a sample are not completely digested. In some embodiments, proteins are subjected to mild denaturation or partial denaturation prior to digestion, such that a particular protein or class of proteins that are more susceptible to denaturation are preferentially digested.

As used herein, the term “protein reducing agent” or “reduction agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl) phosphine hydrochloride (TCEP-HCl), or combinations thereof. A reducing step may be performed in sequence with or concurrent with other sample preparation steps. For example, a reducing step and a denaturing step may be performed simultaneously (by adding a reducing agent while incubating a sample at high temperatures) so that cysteines exposed to the solvent by denaturing can be accessed by the reducing agent.

As used herein, and as well known in the art, the term “liquid chromatography” refers to a process in which a biological and/or chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some embodiments, the sample or eluate can be subjected to any one of the aforementioned chromatographic methods or a combination thereof.

As used herein, and as well known in the art, the term “mass spectrometer” refers to a device capable of identifying specific molecular species and measuring their masses. The term is meant to include any molecular detector in which a polypeptide or peptide may be characterized. A mass spectrometer typically includes three major components: the ion source, the mass analyzer, and the detector. The role of the ion source is to create gas phase ions. Analyte atoms, molecules, or clusters can be transferred into the gas phase and ionized either concurrently (as in electrospray ionization) or through separate processes. The choice of ion source depends on the application.

The mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Acbersold, Selected reaction monitoring-based proteomics: workflows, potential, pitfalls and future directions, 9 NATURE METHODS 555-566 (2012)). MRM is typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimer's disease biomarker candidates, 930 JOURNAL OF CHROMATOGRAPHY B 129-135 (2013)).

SRM/MRM/Selected-ion monitoring (SIM) is a method used in tandem mass spectrometry in which an ion of a particular mass is selected in the first stage of a tandem mass spectrometer and an ion product of a fragmentation reaction of the precursor ion is selected in the second mass spectrometer stage for detection. Examples of triple quadrupole mass spectrometers (TQMS) that can perform MRM/SRM/SIM include but are not limited to QTRAP® 6500 System (Scicx), QTRAP® 5500 System (Sciex), Triple QTriple Quad 6500 System (Sciex), Agilent 6400 Series Triple Quadrupole LC/MS systems, and Thermo Scientific™ TSQ™ Triple Quadrupole system.

In addition to MRM, the choice of peptides can also be quantified through Parallel-Reaction Monitoring (PRM). PRM is the application of SRM with parallel detection of all transitions in a single analysis using a high-resolution mass spectrometer. PRM provides high selectivity, high sensitivity and high-throughput to quantify selected peptides (Q1), and hence quantify proteins. Multiple peptides can be specifically selected for each protein. PRM methodology can use the quadrupole of a mass spectrometer to isolate a target precursor ion, fragment the targeted precursor ion in the collision cell, and then detect the resulting product ions in the Orbitrap mass analyzer. PRM can use a quadrupole time-of-flight (QTOF) or hybrid quadrupole-orbitrap (QOrbitrap) mass spectrometer to carry out the identification of peptides and/or proteins. Examples of QTOF include but are not limited to TripleTOF® 6600 System (Sciex), TripleTOF® 5600 System (Sciex), X500R QTOF System (Sciex), 6500 Series Accurate-Mass Quadrupole Time-of-Flight (Q-TOF) (Agilent) and Xevo G2-XS QT of Quadrupole Time-of-Flight Mass Spectrometry (Waters). Examples of QObitrap include but are not limited to Q Exactive™ Hybrid Quadrupole-Orbitrap Mass Spectrometer (Thermo Scientific) and Orbitrap Fusion™ Tribrid™ (Thermo Scientific).

Non-limiting advantages of PRM include: elimination of most interferences; providing more accuracy and attomole-level limits of detection and quantification; enabling the confident confirmation of the peptide identity with spectral library matching; reducing assay development time since no target transitions need to be preselected; and ensuring UHPLC-compatible data acquisition speeds with spectrum multiplexing and advanced signal processing.

The mass spectrometer in the methods or systems of the present application can be, for example, an electrospray ionization mass spectrometer, nano-electrospray ionization mass spectrometer, or a triple quadrupole mass spectrometer, wherein the mass spectrometer can be coupled to a liquid chromatography system, wherein the mass spectrometer is capable of performing LC-MS (liquid chromatography-mass spectrometry) or LC-PRM-MS (liquid chromatography-parallel reaction monitoring-mass spectrometry) analyses. In some exemplary embodiments, the identification of peptides is performed using PRM-MS.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS2, can be performed by first selecting and isolating a precursor ion (MS1), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The resolution of the tandem mass spectrometry is typically at least about 15,000 (15K) and up to 120,000 (120K). In various embodiments, the resolution may be precisely or about, for example, 15,000, 30,000, 45,000, 60,000, 75,000, 100,000, or 120,000, or a resolution within a range bounded by any two of the foregoing values (e.g., 15K-120K, 30K-120K, 45K-120K, 60K-120K, 75K-120K, 15K-75K, 30K-75K, 45K-75K, or 60K-75K. The isolation window of the tandem mass spectrometry is typically at least about 1 mass-to-charge ratio and up to 18 mass-to-charge ratio. In various embodiments, the isolation window may be precisely or about, for example, 1 mass-to-charge, 2 mass-to-charge, 4 mass-to-charge, 6 mass-to-charge, 8 mass-to-charge, 10 mass-to-charge, 12 mass-to-charge, or 18 mass-to-charge. The isolation window may alternatively be within a range bounded by any two of the foregoing values, e.g., 1-18 mass-to-charge ratio, 2-18 mass-to-charge ratio, 4-18 mass-to-charge ratio, 6-18 mass-to-charge ratio, 8-18 mass-to-charge ratio, 1-12 mass-to-charge ratio, 2-12 mass-to-charge ratio, 4-12 mass-to-charge ratio, 6-12 mass-to-charge ratio, 8-12 mass-to-charge ratio, I-6 mass-to-charge ratio, 2-6 mass-to-charge ratio, or 4-6 mass-to-charge ratio.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited to, identifying the protein, sequencing amino acids of the protein fragments, determining protein sequencing, quantifying the protein, locating post-translational modifications, identifying post translational modifications, or comparability analysis, or combinations thereof.

In some embodiments, the mass spectrometer can use nanoelectrospray or nanospray ionization. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (matrixscience.com), Spectrum Mill (chem.agilent.com), PLGS (waters.com), PEAKS (bioinformaticssolutions.com), Proteinpilot (download.appliedbiosystems.com/proteinpilot), Phenyx (phenyx-ms.com), Sorcerer (sagenresearch.com), OMSSA (pubchem.ncbi.nlm.nih.gov/omssa/), X!Tandem (thegpm.org/TANDEM/), Protein Prospector (prospector.ucsf.edu/prospector/mshome.htm), Byonic (proteinmetrics.com/products/byonic), Sequest (fields.scripps.edu/sequest).

As used herein, the term “algorithm” is a broad term and is used in its ordinary sense, including, but not limited to, the computational processes (for example, programs) involved in transforming information from one state to another, typically done by computer processing. An algorithm is a finite set of instructions carried out in a specific order to perform a particular task. In one embodiment, the algorithm can be an artificial intelligence algorithm. In an exemplary embodiment, the search algorithm is CHIMERYS™ (msaid.de/chimerys). CHIMERYS™ is a cloud native search algorithm that uses accurate predictions of peptide fragment ion intensities and retention times provided by the deep learning framework of INFERYS 2.0.

It is understood that the present invention is not limited to any of the aforesaid protein(s), therapeutic protein(s), antibody(s), recombinant protein(s), host-cell protein(s), protein pharmaceutical product(s), sample(s), AAV(s), virus(es), serotype(s), vector(s), protein alkylating agent(s), protein denaturing agent(s), protein reducing agent(s), digestive enzyme(s), chromatographic method(s), mass spectrometer(s), database(s), bioinformatics tool(s), pH range(s) or value(s), temperature(s), or concentration(s), and any protein(s), therapeutic protein(s), antibody(s), recombinant protein(s), host-cell protein(s), protein pharmaceutical product(s), sample(s), AAV(s), virus(es), serotype(s), vector(s), protein alkylating agent(s), protein denaturing agent(s), protein reducing agent(s), digestive enzyme(s), chromatographic method(s), mass spectrometer(s), database(s), bioinformatics tool(s), pH, temperature(s), or concentration(s) can be selected by any suitable means.

The present invention will be more fully understood by reference to the following examples. They should not, however, be construed as limiting the scope of the invention.

### Examples

### Experimental Methods

### I. Sample Preparation Based on In-solution Digestion

For in-solution digestion, 10 μg of each AAV sample was reduced in 5 mM acetic acid and 1 mM tris(2-carboxyethyl) phosphine (TCEP) at 80° C. for 30 minutes. The samples were then diluted with 8 M urea and 100 mM Tris HCl to achieve a final concentration of 1 M urea. The denatured and reduced samples were subsequently alkylated with 3 mM iodoacetamide (IAM) for 30 minutes in the dark at room temperature. Digestion was performed using trypsin at an enzyme-to-substrate ratio of 1:25 (w/w), with overnight incubation at 37° C. The reaction was quenched by adding TFA to a final concentration of 0.5% TFA. The samples were then desalted using commercial desalting tips, following the manufacturer's protocols. After desalting, the samples were dried and reconstituted in 0.1% FA for LC-MS analysis, where TFA=trifluoroacetic acid and FA=formic acid.

### II. Sample Preparation Based on SP3 (Paramagnetic) Beads

The protein concentration of AAV samples was measured by the BCA protein assay following the manufacturer's protocol prior to sample processing. Two stock Sera-Mag™-magnetic beads (50 mg/mL) were combined in a 1:1 ratio, washed with water, and then diluted to 25 mg/mL. For AAV sample inputs of 1 and 2.5 μg, the samples were diluted to a final volume of 20 μL using 8 M urea. For sample inputs of 5 and 10 μg, the samples were diluted to a final volume of 50 μL using 8 M urea. Dithiothreitol (DTT) was added to achieve a final concentration of 10 mM, and the mixture was incubated at 50° C. for 30 min for reduction. Samples were then alkylated with 25 mM IAM for 30 min in the dark at room temperature. Bead mixtures were added at a protein-to-bead ratio of 1:10 (w/w). To induce protein binding to the beads, an equal volume of EtOH was added to the sample to achieve a final concentration of 50% EtOH. The binding mixture was incubated in at 24° C. for 10 min in a commercial thermo-mixer at 1000 rpm. On a magnetic rack, the supernatant was removed and discarded. The samples were then washed three times with 180 μL of 80% EtOH. Trypsin enzyme was added to each sample at an enzyme-to-substrate ratio of 1:25 (w/w) in 50 μL of 50 mM Tris-HCl (pH=8). The proteins were digested overnight at 37° C. in a commercial thermo-mixer at 1000 rpm. Post-digestion, the sample was centrifuged at 20,000 g for 1 min. The supernatant was then collected on a magnetic rack and transferred to a new tube. The reaction was terminated by adding FA to a final concentration of 0.5% FA. The samples were dried and reconstituted in 0.1% FA for LC-MS analysis.

### III. Sample Preparation Based on Filter-Aided Sample Preparation (FASP) Method

The AAV samples of 1 to 10 μg were transferred to a 10 kDa MWCO filter. To deplete the surfactants, 200 μL of 8 M urea was added, followed by centrifugation at 14,000 g for 15 minutes. For reduction, 50 μL of 8 M urea containing 10 mM DTT was added to the samples, and the samples were then incubated at 50° C. for 30 minutes. Subsequently, a final concentration of 25 mM IAM was added, and samples were incubated for 30 minutes in the dark at room temperature. The samples were then washed with 100 μL of 50 mM Tris-HCl through centrifugation for 10 minutes at 14,000 g. Trypsin was added to each sample at an enzyme-to-substrate ratio of 1:25 (w/w) in 50 μL of 50 mM Tris-HCL (pH=8), followed by overnight incubation at 37° C. After digestion, the sample filter was centrifuged until dry and washed one time by adding another 50 μL of 0.1% FA buffer. The flow-through was collected and dried, then reconstituted in 0.1% FA prior to LC-MS analysis.

### IV. HCP Sample Preparation of NISTmAb

The ultra-low trypsin digestion method was utilized for the preparation of NISTmAb HCP sample, following a previously established protocol (S. Nic et al., Anal. Chem. 2021, 93 (10), 4383-4390). Briefly, 1 mg of NISTmAb was desalted, after which the buffer was exchanged for a 50 mM Tris-HCl buffer. This was done using 10 kDa MWCO commercial centrifugal filters, resulting in a final mAb concentration of 2 mg/mL. The sample was then digested overnight at 37° C. at a trypsin-to-substrate ratio of 1:10000 (w/w). After digestion, TCEP was added to achieve a final concentration of 5 mM. The samples were then acidified to a final concentration of 0.1% FA and centrifuged at 14,000 g for 10 minutes. The supernatant was further filtered using a 10 kDa MW cutoff filter, and the flow-through was dried. The samples were resuspended in 0.1% FA prior to LC-MS analysis.

### V. Formula for HCPs Quantification

The top three quantification methods were used for HCP protein quantification. Prior to sample processing, 100 μg of Heavy labeled PLBD2 protein were spiked into each AAV sample with a final mass ratio of 40 ppm, which was calculated based on the total protein amount of AAV samples (2.5 μg). The host cell proteins' relative abundance was calculated based on the relative mass ratio (ppm) with spiked—in heavy labeled PLBD2 proteins. First, the mass ratio was calculated using the average of the top three peptide abundance and the molecular weight (MW) of the Host Cell Proteins (HCPs) (e.g., protein A), and the spiked—in heavy-labeled PLBD2 (PLBD2\hat{ }). This calculation is detailed in Equation (1) below. Subsequently, the mass ratio (ppm) of the HCPs was computed using Equation (2), and the concentration (ng/ml) of the HCPs was determined using Equation (3).

$$Mass ratio = \frac{Average of top 3 peptides abundance of protein A * MW of Protein A}{Average of top 3 peptides abundance of spike - in PLBD 2 \hat{} * MW of PLBD 2 \hat{}} \qquad ( 1 )$$

$$Mass ratio ( ppm ) = Mass ratio * 40 ppm PLBD 2 \hat{} \qquad ( 2 )$$

$$Concentration ( ng / mL ) = \frac{Mass Ratio * 0.1 ng PLBD 2 \hat{}}{AAV initial volume ( mL )} \qquad ( 3 )$$

### Example 1. Optimization of Sample Preparation

Current methods for HCP identification include direct digestion, immunoprecipitation, native digestion, and the use of molecular weight cutoff filters. However, there are several challenges and shortcomings with the current methods for HCP identification, especially while profiling AAVs for HCP impurities. Some of the challenges and shortcomings include, for example, low product yields and surfactants present in the final AAV products.

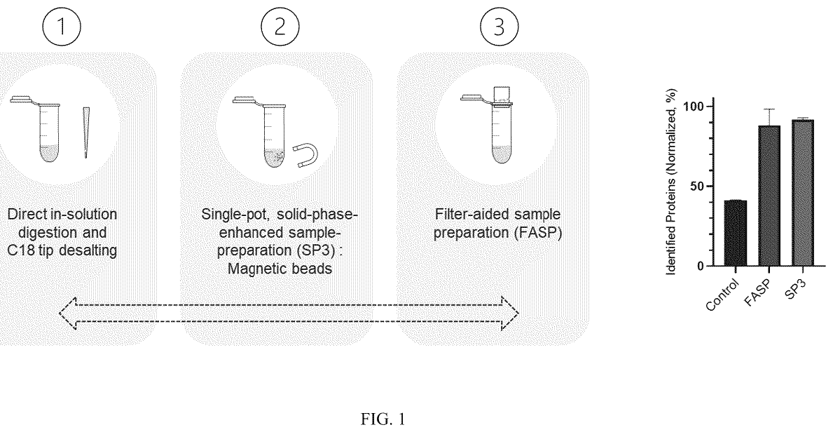

To overcome the small and limited samples, three sample preparation methods were compared for processing minimal amounts of AAVs in a sample, as shown in FIG. 1. These methods included a control method with direct in-solution digestion, single-pot, solid phase-enhanced sample-preparation (SP3) magnetic beads and Filter-Aided Sample Preparation (FASP). A significant increase in the percentage of identified proteins was observed in the FASP and SP3 sample preparation methods.

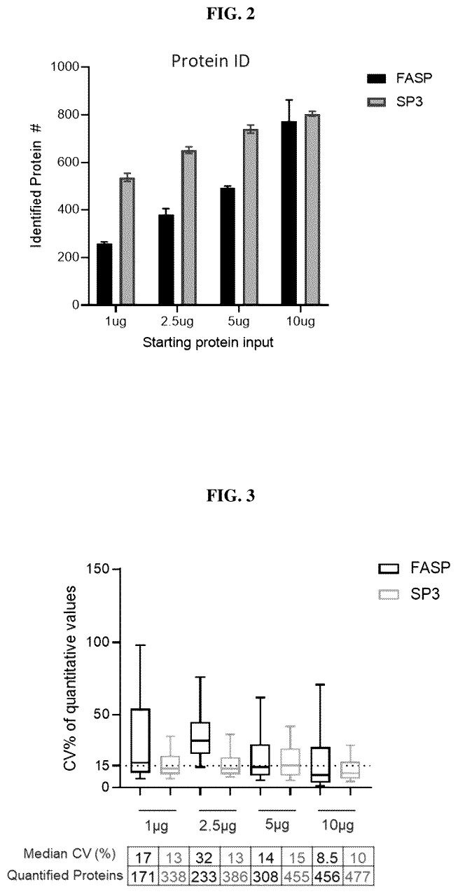

The SP3 and FASP methods were compared using varying amounts of protein input. The number of identified proteins was greater for the SP3 sample preparation method as compared to the FASP, as shown in FIG. 2. In addition, the SP3 method was more reproducible and has less sample loss when utilizing low protein input at the 1 μg, 2.5 μg or 5 μg concentrations, as shown in FIG. 3. To maximize HCP detection, the optimization of mass spectrometry was evaluated.

### Example 2. Optimization of Mass Spectrometry

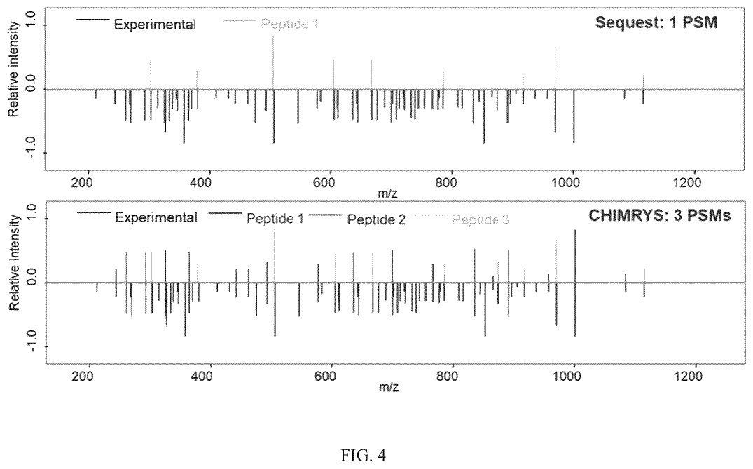

To further improve the identification of HCPs, the mass spectrometry data acquisition and analysis was optimized. The wide window acquisition (WWA) parameters that were optimized included MS2 resolution, isolation window, maximum injection time, and Automatic Gain Control. The WWA method allows for better separation of chimeric spectra, and improves the coverage of low abundant peptides present in the sample. FIG. 4 shows the spectrum of a traditional search engine using a peptide-to-spectrum matching (PSM) of one (top panel) as compared to the WWA spectrum using CHIMERYS™ and a PSM of four (bottom panel). The chimeric spectrum shows additional ions with similar mass and retention time that were not identified in the traditional spectrum.

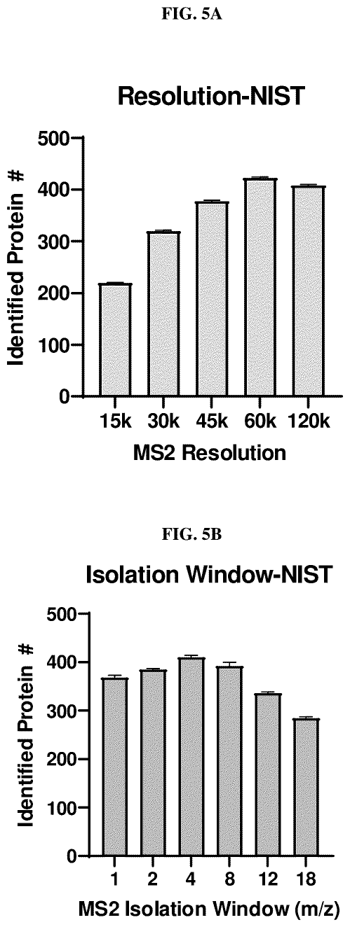

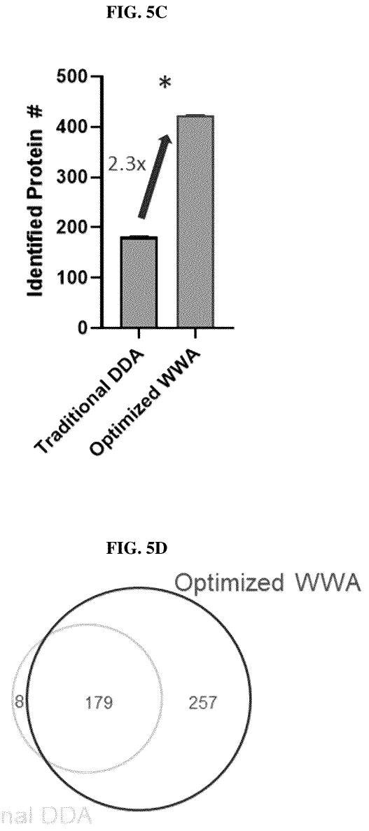

When comparing the optimization of resolution, it is apparent that an increase in the number of proteins identified correlates with a higher resolution. The 60K resolution provided the highest number of proteins identified and is necessary for the deconvolution of chimeric spectra, as shown in FIG. 5A. In addition to resolution, the MS2 isolation window was optimized. A wide isolation window of 4 mass-to-charge ratio yielded the highest number of identified proteins, as shown in FIG. 5B. When comparing traditional DDA to the optimized WWA of the present method, a 2.3-fold increase in the number of proteins identified was observed, as shown in FIG. 5C. FIG. 5D illustrates the results of the analysis, where out of the 444 HCPs identified, 257 new proteins were identified using the optimized WWA parameters, 179 were both identified using the method of the present invention and traditional DDA, and only 8 were identified alone by traditional DDA. Using this proof-of-concept with the NISTmAb HCPs, the method of the present invention was optimized using AAVs.

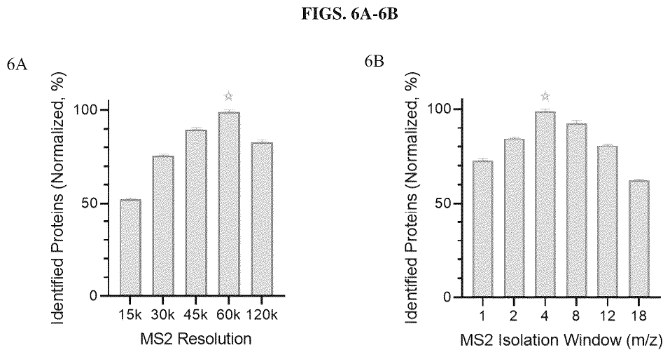

As with the AAVs HCPs, the MS2 resolution yielding the highest percentage of proteins identified was 60K, as shown in FIG. 6A. In addition to resolution, the MS2 isolation window of 4 mass-to-charge ratio yielded the highest percentage of proteins identified, as shown in FIG. 6B.

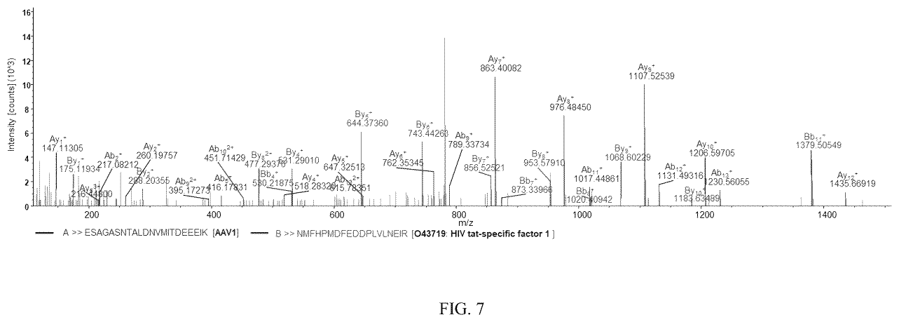

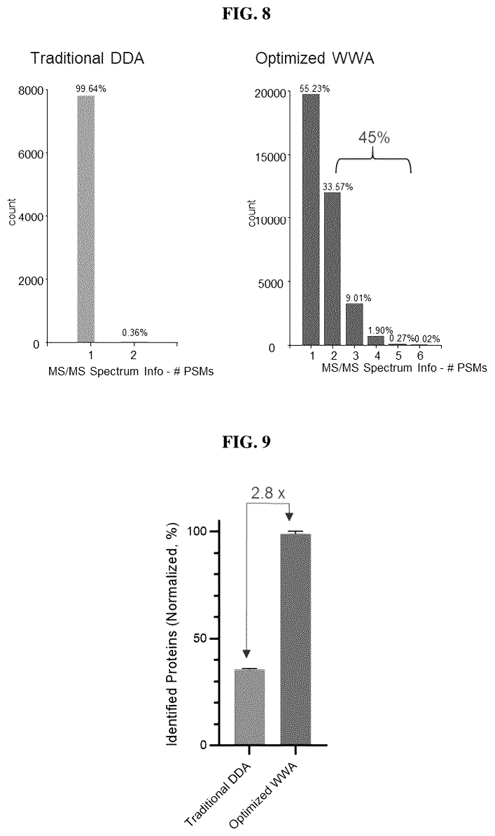

Using optimized parameters of the present invention, the chimeric spectra for AAV identified several low abundance peptides as shown in FIG. 7. Further, the optimized method using AAV was compared to that of traditional DDA as shown in FIG. 8. Approximately 45% of spectra are chimeric, as observed in the optimized WWA, with the PSM at 2 or greater. This yielded a 2.8-fold increase in the percentage of identified proteins as shown in FIG. 9, when compared to the traditional DDA approach, using SEQUEST search.

### Example 3. Case Studies using different AAV serotypes

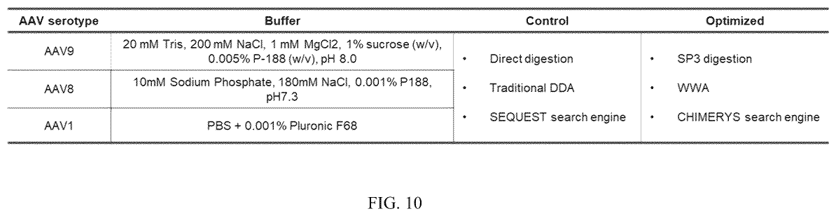

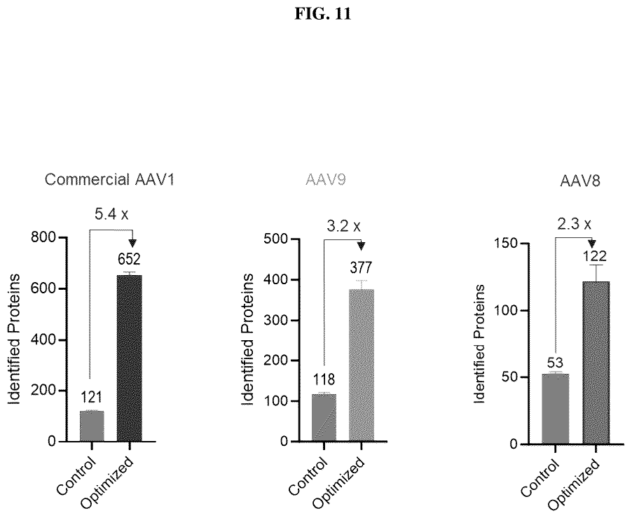

The optimized methods described in Example 1 and Example 2 were used with several different AAV serotypes and compared to the traditional DDA method of analysis. A significant increase in the number of identified proteins was observed as compared to the traditional method, as shown in FIG. 10. This increase was observed across all AAV serotypes tested. FIG. 11 shows an increase in protein identification of 5.4-fold increase for AAV1,3.2-fold increase for AAV9 and 2.3-fold increase for AAV8. For example, using a commercially available AAV1, 121 HCPs were identified under the most stringent conditions (at least 2 unique peptides per protein, 1% peptide false discovery rate (FDR) and 1% protein FDR). In comparison, using the method of the present invention, 652 HCPs were identified, demonstrating the effectiveness of the optimization.

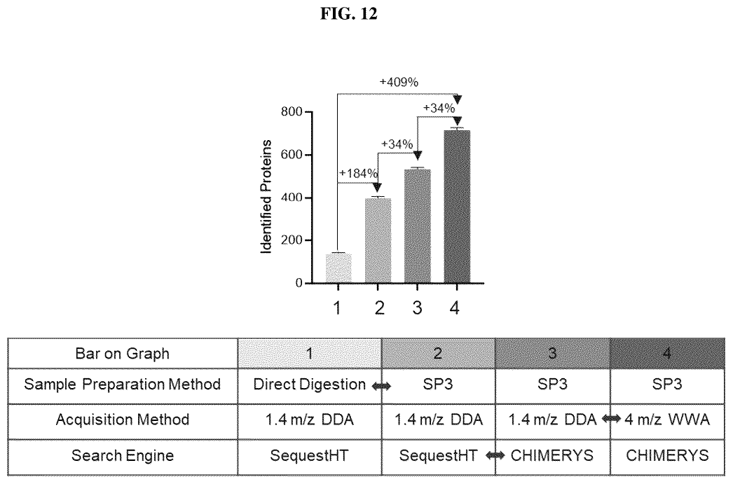

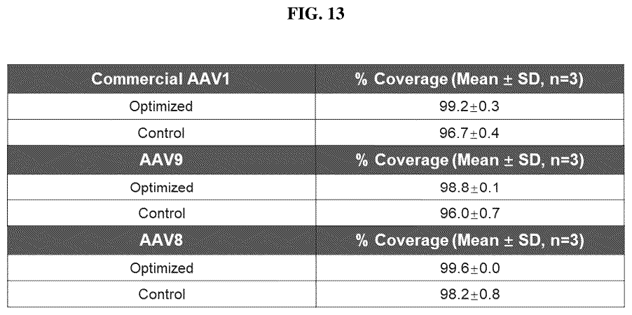

In addition, a step-wise approach observing the improvement by replacing one component is shown in FIG. 12. FIG. 12 illustrates the change in the number of HCPs identified using the traditional method (column 1). When compared to the number of HCPs identified when the sample preparation method was replaced with SP3, an increase of 184% was observed. When the search algorithm was replaced, in combination with the SP3 sample preparation method, an increase in HCP identification of 34% was observed. Finally, when the acquisition method was increased to 4 mass-to-charge ratio using WWA from the traditional 1.4 mass-to-charge ratio DDA method, an additional 34% increase in HCP identification was observed. In total, an increase of 409% HCP identification was observed using the optimized method of the present invention. Finally, high protein coverage was observed using the optimized methods of the present invention and Byonic software, as shown in FIG. 13.

### Example 4. Quantification of HCPs

The optimized methods were used to quantify contaminant viral proteins along with human HCPs. Heavy-label PLBD2 (PLBD2\hat{ }) was spiked into the sample before processing at a mass ratio of 40 ppm. The mass ratio of host cell proteins was calculated by using the average of the top three peptides abundance of protein A multiplied by the molecular weight of protein A and multiplied by 40, divided by the average of top 3 peptides abundance of PLBD2\hat{ } multiplied by molecular weight of PLBD2\hat{ }. The concentration of host cell proteins (ng/ml) was calculated by the mass ratio multiplied by the mass of PLBD2\hat{ }, divided by the AAV initial volume (mL).

$$Mass ratio ( ppm ) = \frac{Average of top 3 peptides abundance of protein A * MW of protein A * 40 *}{Average of top 3 peptides abundance of spike - in PLBD 2 \hat{} * MW of PLBD 2 \hat{}}$$

$$Concentration ( ng / mL ) = Mass ratio * Mass of PLBD 2 \hat{} / AAV initial volume ( mL )$$

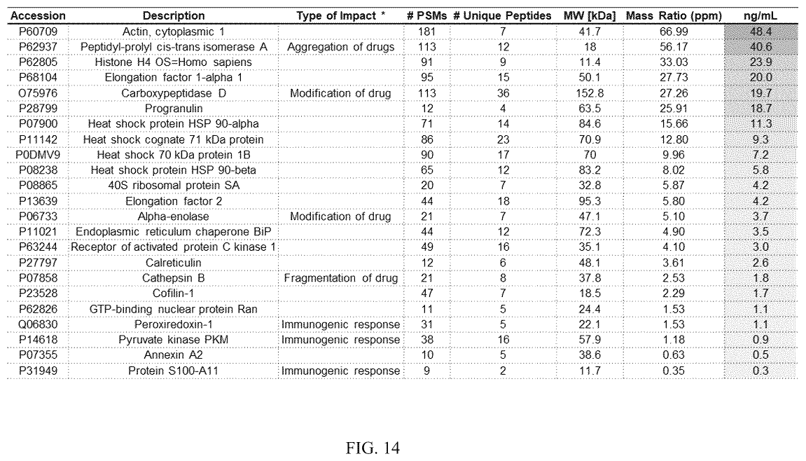

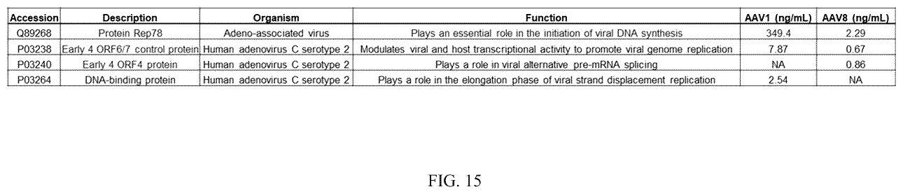

As shown in FIG. 14, many high-risk HCPs were quantified using the optimized methods of the present invention. In addition, several viral protein contaminants were identified, including Rep78, E4 and E2 protein, as shown in FIG. 15. FIG. 15 shows viral protein contaminants identified in AAVs, according to an exemplary embodiment. In total, six viral proteins, including Rep78, E4, and E2 protein, from human adenovirus were identified in all three AAV serotypes, at least 2 unique peptides per protein and three were quantifiable in all three serotypes. Additionally, Rep78 was detected in a relatively high abundance, at about 100 ppm molar ratio in AAV1 samples.

FIG. 15 shows viral protein contaminants identified in AAVs, according to an exemplary embodiment. Besides HCPs and AAV capsid proteins, AAV products may also contain viral protein impurities that are expressed from the plasmids during the production of AAV capsids, which are often overlooked during impurity analysis. In theory, these foreign viral proteins may pose a higher immunogenic risk compared to HCPs originating from human HEK293 cells. By searching the raw data against a more comprehensive database, which was constructed by concatenating the human database with AAV-associated protein database, four process-related viral proteins were identified and three were quantified, including Rep 78, E4, and DNA-binding (Early 2A) protein. Among them, the Rep 78 protein is known to be expressed from the Rep-Cap plasmid during AAV production, which exhibited a relatively high abundance. E4 and E2 proteins that were expressed from helper plasmid were also identified although at much lower abundances. These findings highlight the potential presence of process-related viral protein impurities in AAV products.

### CONCLUSIONS

The above examples provide a comprehensive evaluation of AAV HCP methods using several approaches. By one approach, various sample preparation methods were compared in which the sample amount was varied from 1 to 10 μg AAV. These methods included a control method with direct in-solution digestion, Filter-Aided Sample Preparation (FASP), and SP3 methods. These findings demonstrate that, when using a higher input at 10 μg, both FASP and SP3 methods resulted in more HCPs than the control method. However, when using a lower input at 1 μg, 2.5 μg, or 5 μg, the SP3 method achieved a much greater depth and reproducibility. Second, the wide window acquisition (WWA) method was optimized using AAV HCP digests. Some of the key parameters focused on included MS2 resolution, isolation window, maximum injection time, and Automatic Gain Control. By these optimized acquisition methods in combination with the CHIMERYS™ search engine, a 2.8-fold increase in HCP identification was achieved compared to traditional DDA with SEQUEST search. Third, different AAV serotypes were analyzed using a combination of optimized methods, including SP3 digestion with WWA. These optimized methods led to an increase in the identification of HCP numbers by 5.4, 3.2, and 2.3-fold for AAV1, AAV8, and AAV9 samples, respectively. For example, in a commercially available AAV1 product, using control methods, 121 HCPs were identified under the most stringent control conditions (at least 2 unique peptides per proteins, 1% peptide FDR, and 1% protein FDR). However, using the present optimized approach, 652 HCPs were identified in the AAV1 samples, which demonstrates the effectiveness of the present optimized method. Fourth, quantification of contaminant viral proteins was achieved along with human HCPs. Several viral proteins, including Rep78, E4, and E2 protein, were identified. Significantly, Rep78 was detected in relatively high abundance, at around a 100 ppm molar ratio in AAV1 samples. These viral proteins have the potential to initiate an immune response. Lastly, the WWA methods were also used in mAb HCPs analysis. In tests that were conducted using NISTmAb, a significant increase in HCP identifications by 2.3 folds was achieved. In conclusion, the optimized methods using SP3 digestion, WWA, and CHIMERYS are straightforward to implement, highly sensitive, and reproducible. These methods provide significant benefits to AAV HCP analysis and quality control.

While there have been shown and described what are at present considered the preferred embodiments of the invention, those skilled in the art may make various changes and modifications which remain within the scope of the invention defined by the appended claims.

1. A method of identifying, quantifying, and/or characterizing at least one host cell protein (HCP) impurity in a sample containing a biotherapeutic, comprising:
(a) treating said sample to enzymatic digestion to produce a peptide digest;
(b) subjecting said peptide digest to paramagnetic beads to form a purified peptide digest; and
(c) subjecting said purified peptide digest to liquid chromatography-mass spectrometry (LC-MS) analysis utilizing wide window acquisition to identify, quantify, and/or characterize said at least one HCP impurity.

2. The method of claim 1, wherein said mass spectrometry is tandem mass spectrometry.

3. The method of claim 2, wherein the resolution of the tandem mass spectrometry is about 15 K to about 120 K.

4. The method of claim 3, wherein the resolution of the tandem mass spectrometry is about 60 K.

5. The method of claim 2, wherein the isolation window of the tandem mass spectrometry is between about 1 mass-to-charge ratio and about 18 mass-to-charge ratio.

6. The method of claim 5, wherein the isolation window of the tandem mass spectrometry is about 4 mass-to-charge ratio.

7. The method of claim 1, wherein the sample amount is from about 1 μg to about 10 μg.

8. The method of claim 7, wherein the sample amount is about 2.5 μg.

9. The method of claim 1, wherein the biotherapeutic comprises a viral particle.

10. The method of claim 9, wherein the viral particle comprises at least one AAV vector.

11. The method of claim 10, wherein said at least one AAV vector comprises a serotype selected from the group comprising AAV1, AAV2, AAV3, AAV4, AAV5, AAV6, AAV7, AAV8, AAV9, AAV10, AAV11, AAV12, variations thereof and combinations thereof.

12. The method of claim 1, wherein the biotherapeutic comprises a monoclonal antibody (mAb).

13. The method of claim 1, wherein the HCP impurity is a peptide impurity.

14. The method of claim 13, wherein said peptide impurity identification is enhanced by a search algorithm.

15. The method of claim 14, wherein said search algorithm is CHIMERYS™.

16. The method of claim 1, wherein the paramagnetic beads are a single-pot, solid-phase enhanced sample-preparation (SP3).

17. A method of identifying, quantifying, and/or characterizing at least one host cell protein (HCP) impurity in a sample containing a biotherapeutic, comprising:
(a) treating said sample to enzymatic digestion to produce a peptide digest;
(b) subjecting said peptide digest to filter-aided sample preparation to form a purified peptide digest; and
(c) subjecting said purified peptide digest to liquid chromatography-mass spectrometry (LC-MS) analysis utilizing wide window acquisition to identify, quantify, and/or characterize said at least one HCP impurity.

18. The method of claim 17, wherein said mass spectrometry is tandem mass spectrometry.

19. The method of claim 18, wherein the resolution of the tandem mass spectrometry is about 15 K to about 120 K.

20. The method of claim 19, wherein the resolution of the tandem mass spectrometry is about 60 K.

21. The method of claim 18, wherein the isolation window of the tandem mass spectrometry is between about 1 mass-to-charge ratio and about 18 mass-to-charge ratio.

22. The method of claim 21, wherein the isolation window of the tandem mass spectrometry is about 4 mass-to-charge ratio.

23. The method of claim 17, wherein the sample amount is from about 1 μg to about 10μ g.

24. The method of claim 23, wherein the sample amount is about 2.5 μg.

25. The method of claim 17, wherein the biotherapeutic comprises a viral particle.

26. The method of claim 25, wherein the viral particle comprises at least one AAV vector.

27. The method of claim 26, wherein said at least one AAV vector comprises a serotype selected from the group comprising AAV1, AAV2, AAV3, AAV4, AAV5, AAV6, AAV7, AAV8, AAV9, AAV10, AAV11, AAV12, variations thereof and combinations thereof.

28. The method of claim 17, wherein the biotherapeutic comprises a monoclonal antibody (mAb).

29. The method of claim 17, wherein the HCP impurity is a peptide impurity.

30. The method of claim 17, wherein said peptide impurity identification is enhanced by a search algorithm.

31. The method of claim 30, wherein said search algorithm is CHIMERYS™.

32. A method of identifying, quantifying, and/or characterizing at least one host cell protein (HCP) impurity in a sample containing a monoclonal antibody (mAb), comprising:
(a) treating said sample to enzymatic digestion to produce a peptide digest; and
(b) subjecting said purified peptide digest to liquid chromatography-mass spectrometry (LC-MS) analysis utilizing wide window acquisition to identify, quantify, and/or characterize said at least one HCP impurity.

33-42. (canceled)

43. A method of identifying, quantifying, and/or characterizing at least one host cell protein (HCP) impurity in a sample containing AAV vectors, comprising:
(a) treating said sample to enzymatic digestion to produce a peptide digest;
(b) subjecting said peptide digest to single-pot, solid-phase enhanced sample-preparation paramagnetic beads to form a purified peptide digest; and
(c) subjecting said purified peptide digest to liquid chromatography-mass spectrometry (LC-MS) analysis utilizing wide window acquisition to identify, quantify, and/or characterize said at least one HCP impurity.

44-54. (canceled)
